(19) United States
(12) Patent Application Publication
(10) Pub. No.: US 2021/0024582 A1
(43) Pub. Date: Jan. 28, 2021

(54) CHIMERIC TIM-3 FUSION PROTEIN

(71) Applicant: H. Lee Moffitt Cancer Center and Research Institute, Inc., Tampa, FL (US)

(72) Inventors: Alan List, Tampa, FL (US); Sheng Wei, Tampa, FL (US)

(21) Appl. No.: 16/982,159

(22) PCT Filed: Mar. 28, 2019
(86) PCT No.: PCT/US2019/024550

§ 371 Date: Sep. 18, 2020

Related U.S. Application Data
(60) Provisional application No. 62/649,887, filed on Mar. 29, 2018.

(51) Int. Cl. C07K 14/00

(52) U.S. Cl. CPC C07K 14/001; C07K 2319/02

(57) ABSTRACT

Disclosed herein is a chimeric polypeptide, comprising at least a portion of the TIM3 receptor ectodomain and a fusion moiety for solubilizing the TIM3 receptor. Also disclosed is a method for treating a hematological cancer in a subject that involves administering to the subject a therapeutically effective amount of the disclosed chimeric polypeptide. Also disclosed is a method for enhance hematopoiesis in a subject, comprising administering to the subject a therapeutically effective amount of the disclosed chimeric polypeptide.

### CROSS-REFERENCE TO RELATED APPLICATIONS

This application claims benefit of U.S. Provisional Application No. 62/649,887, filed Mar. 29, 2018, which is hereby incorporated herein by reference in its entirety.

### SEQUENCE LISTING

This application contains a sequence listing filed in electronic form as an ASCII.txt file entitled “320803_2220_Sequence_Listing_ST25” created on Mar. 29, 2019. The content of the sequence listing is incorporated herein in its entirety.

### BACKGROUND

Acute Myeloid Leukemia (AML) and Myelodysplastic syndrome (MDS) arise from accumulation of multiple stepwise genetic and epigenetic changes in hematopoietic stem cells (HSC) and/or committed progenitors. A series of transforming events can initially give rise to pre-leukemia stem cells (pre-LSC) as well as fully transformed leukemia stem cells (LSC), both of which need to be targeted in strategies aimed at curing these diseases.

### SUMMARY

Disclosed herein is a chimeric polypeptide, comprising at least a portion of the TIM3 receptor ectodomain and a fusion moiety for solubilizing the TIM3 receptor. For example, the fusion moiety can be an Fc portion of an immunoglobulin. In some embodiments, the TIM3 ectodomain comprises at least the IgV domain. In some embodiments, the TIM3 ectodomain further comprises the mucin domain, the transmembrane domain, the cytoplasmic domain, or any combination thereof. In some embodiments, the TIM3 ectodomain lacks the mucin domain, the transmembrane domain, the cytoplasmic domain, or any combination thereof. The fusion protein can further comprise a signal peptide for secretion of the protein.

In some cases, the polypeptide is defined by the formula:

SP-IgV-Mucin-TM-Cyto-Fc,

wherein “SP” represents a signal peptide,

wherein “IgV” represents an IgV domain of TIM3,

wherein “Mucin” represents an optional mucin domain of TIM3,

wherein “TM” represents an optional transmembrane domain of TIM3,

wherein “Cyto” represents an optional cytoplasmic domain of TIM3,

wherein “Fc” represents an Fc portion of an immunoglobulin, and

wherein “-” represents a linker peptide or peptide bond.

In some cases, the polypeptide is defined by the formula:

SP-IgV-Mucin-TM-Cyto-Fc,

wherein “SP” represents a signal peptide,

wherein “IgV” represents an IgV domain of TIM3,

wherein “Mucin” represents a mucin domain of TIM3,

wherein “TM” represents an transmembrane domain of TIM3,

wherein “Cyto” represents a cytoplasmic domain of TIM3,

wherein “Fc” represents an Fc portion of an immunoglobulin, and

wherein “-” represents a linker peptide or peptide bond.

In some embodiments, the TIM-3 ectodomain has the amino acid sequence:

MFSHLPFDCVLLLLLLLLTRSSEVEYRAEVGQNAYLPCFYTPAAPGNLVPVC WGKGACPVFECGNVVLRTDERDVNYWTSRYWLNGDFRKGDVSLTIENVTLADSGI YCCRIQIPGIMNDEKFNLKLVIKPAKVTPAPTRQRDFTAAFPRMLTTRGHGPAETQT LGSLPDINLTQISTLANELRDSRLANDLRDSGATIRIGIYIGAGICAGLALALIFGALIFK WYSHSKEKIQNLSLISLANLPPSGLANAVAEGIRSEENIYTIEENVYEVEEPNEYYCY VSSRQQPSQPLGCRFAMP (SEQ ID NO:1), or an amino acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1.

In some embodiments, the TIM-3 ectodomain has the amino acid sequence:

MFSHLPFDCVLLLLLLLLTRSSEVEYRAEVGQNAYLPCFYTPAAPGNLVPVC WGKGACPVFECGNVVLRTDERDVNYWTSRYWLNGDFRKGDVSLTIENVTLADSGI YCCRIQIPGIMNDEKFNLKLVIKPGGSGGSGGSGALIFKWYSHSKEKIQNLSLISLAN LPPSGLANAVAEGIRSEENIYTIEENVYEVEEPNEYYCYVSSRQQPSQPLGCRFAMP (SEQ ID NO:2), or an amino acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:2.

In some embodiments, the signal peptide is a TIM-3 signal peptide. In some embodiments, the signal peptide has the amino acid sequence: MFSHLPFDCVLLLLLLLLTRS (SEQ ID NO:3), or an amino acid sequence having at least 65%, 70%, 75%, 80%, 85%, 90%, 95% sequence identity to SEQ ID NO:3.

In some embodiments, the IgV domain has the amino acid sequence: SEVEYRAEVGQNAYLPCFYTPAAPGNLVPVCWGKGACPVFECGNVVLRTDERDV NYWTSRYWLNGDFRKGDVSLTIENVTLADSGIYCCRIQIPGIMNDEKFNLK (SEQ ID NO:4), or an amino acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:4 capable of binding a TIM-3 ligand.

In some embodiments, the mucin domain has the amino acid sequence: LVIKPAKVTPAPTRQRDFTAAFPRMLTTRGHGPAETQTLGSLPDINLTQISTLANELR DSRLANDLRDSGATIR (SEQ ID NO:5), or an amino acid sequence having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:5.

In some embodiments, the transmembrane domain has the amino acid sequence: IGIYIGAGICAGLALALIFGA (SEQ ID NO:6), or an amino acid sequence having at least 65%, 70%, 75%, 80%, 85%, 90%, 95% sequence identity to SEQ ID NO:6.

In some embodiments, the cytoplasmic domain has the amino acid sequence: LIFKWYSHSKEKIQNLSLISLANLPPSGLANAVAEGIRSEENIYTIEENVYEVEEPNEY YCYVSSRQQPSQPLGCRFAMP (SEQ ID NO:7), or an amino acid sequence having at least 65%, 70%, 75%, 80%, 85%, 90%, 95% sequence identity to SEQ ID NO:7.

In some embodiments, the linker has the amino acid sequence: GGSGGSGGS (SEQ ID NO:8).

Fc domains for use in fusion proteins are known in the art and available from, for example, InvivoGen (San Diego, Calif.). They can be purchased as cloning plasmids with multiple cloning sites (MCS) to facilitate cloning. In some cases, the plasmids contain an IL2 signal sequence for proteins that are not naturally secreted.

Also disclosed is a pharmaceutical composition comprising a molecule disclosed herein in a pharmaceutically acceptable carrier.

Also disclosed is a method for treating a cancer, such as a hematological cancer, in a subject that involves administering to the subject a therapeutically effective amount of a disclosed pharmaceutical composition. The cancer can in some embodiments be any cancer that expresses Tim3 or a Tim3 ligand (e.g. galectin-9). In some cases, the cancer comprises a myelodysplastic syndrome (MDS). For example, the cancer can be non-del(5q) MDS. In some cases, the cancer comprises an Acute Myeloid Leukemia (AML).

Also disclosed is a method for enhance hematopoiesis in a subject, comprising administering to the subject a therapeutically effective amount of a disclosed pharmaceutical composition.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates the structure of a chimeric sTim3 receptor and a chimeric fTim3 receptor.

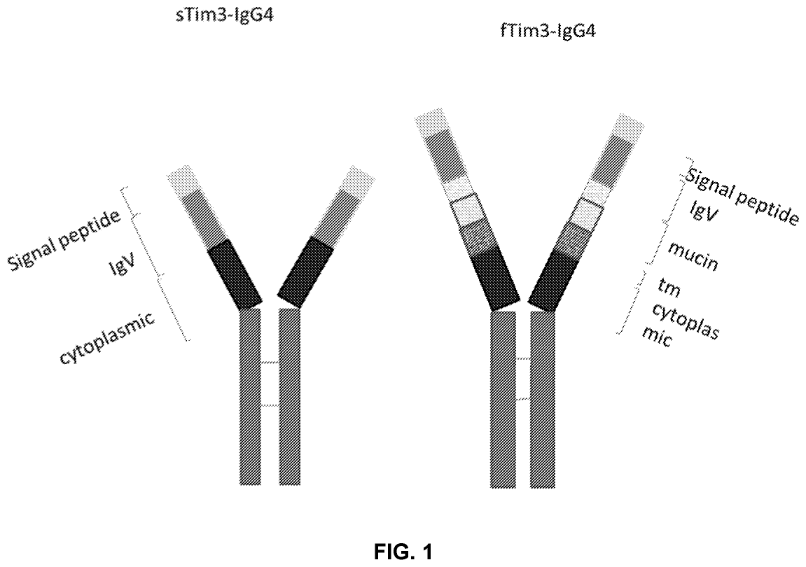

FIG. 2 illustrates the structure of a Tim3 receptor that is glycosylated and anchored in a cell membrane, comprising an IgV-like domain, a mucin domain, a to transmembrane domain, and an intracellular tail.

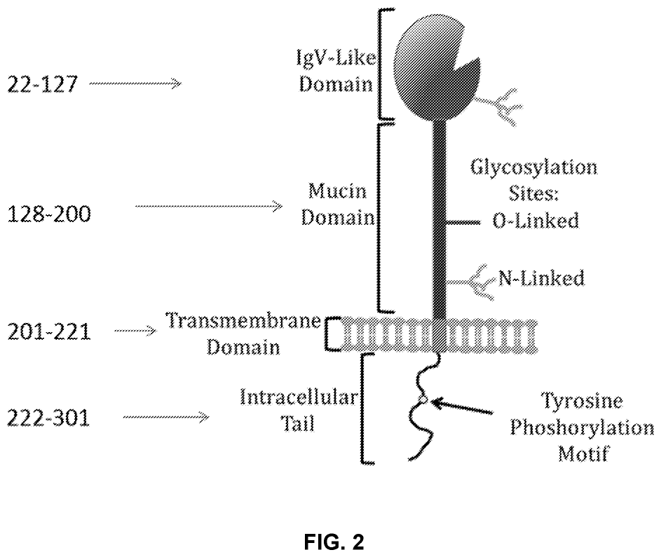

FIG. 3 is a sequence alignment of sTIM-3 (SEQ ID NO:2) and fTIM-3 (SEQ ID NO:1) chimeric receptors.

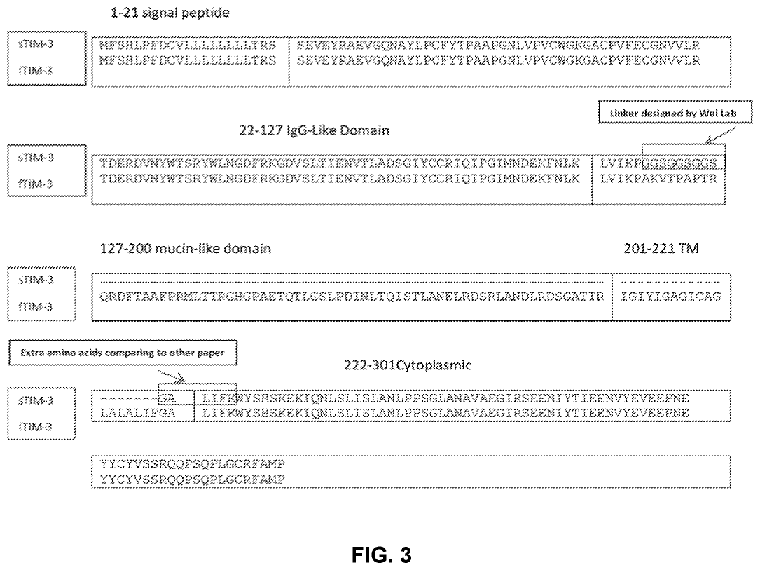

FIG. 4 is a bar graph showing results of a hematopoietic colony forming assay after treatment to measure the restoration of specific progenitors after treatment with IgG4 control or sTIM3 fusion or fTim3 fusion. Figure shows duplicate determinations of one patient specimen.

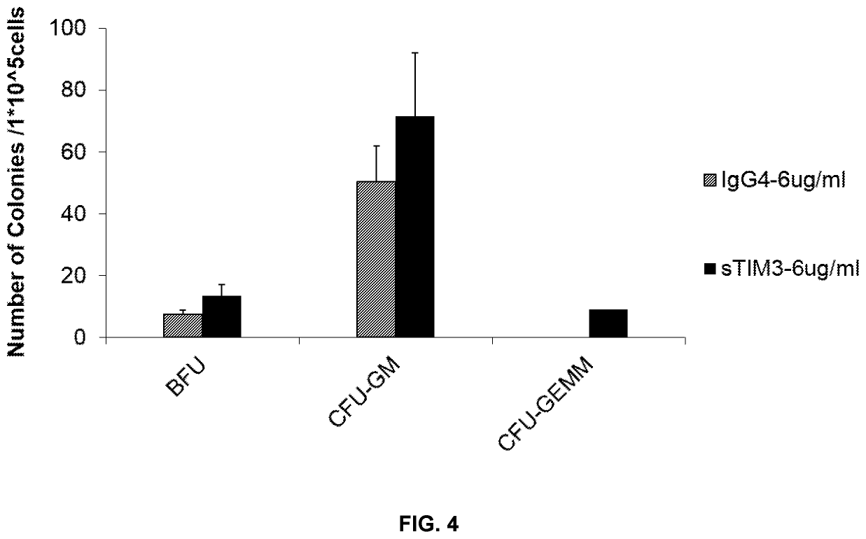

FIG. 5 is a bar graph showing results of a hematopoietic colony forming assay after treatment to measure the restoration of specific progenitors after treatment with IgG4 control or sTIM3 fusion or fTim3 fusion. Figure shows duplicate determinations of one patient specimen.

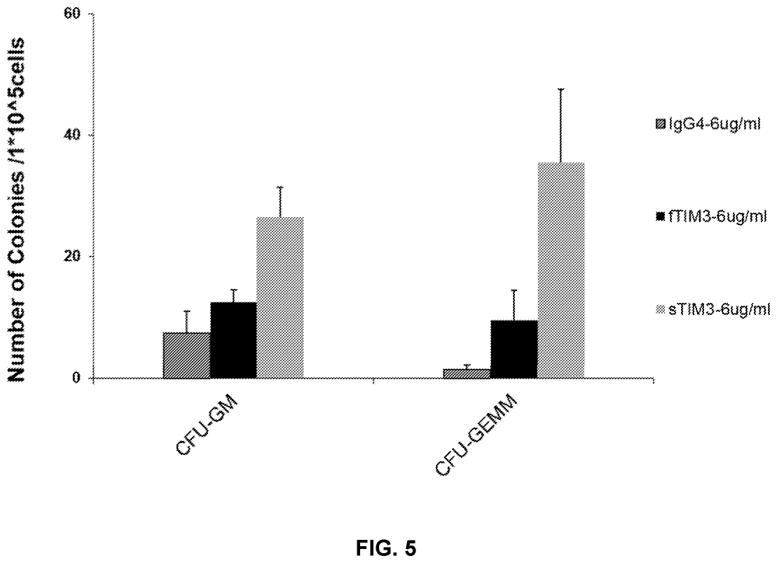

FIG. 6 is a bar graph showing results of a hematopoietic colony forming assay after treatment to measure the restoration of specific progenitors after treatment with IgG4 control or sTIM3 fusion or fTim3 fusion. Figure shows duplicate determinations of one patient specimen.

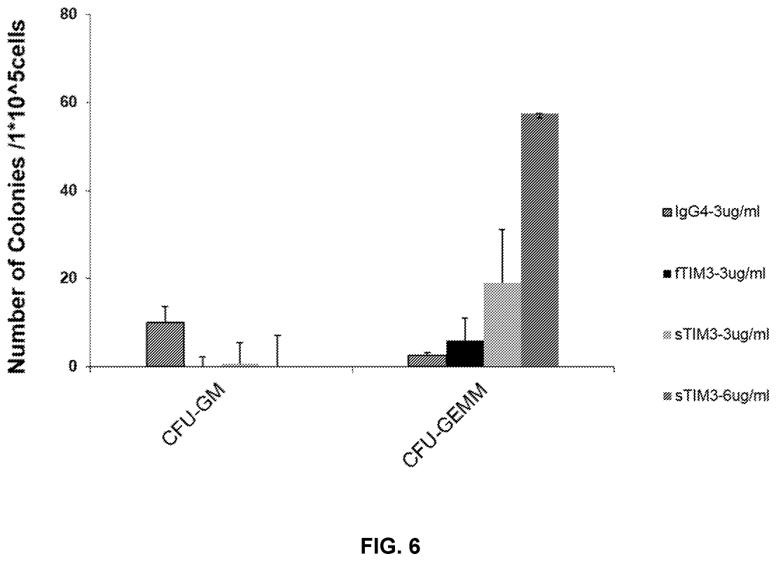

FIG. 7 is a bar graph showing results of a hematopoietic colony forming assay after treatment to measure the restoration of specific progenitors after treatment with IgG4 control or sTIM3 fusion or fTim3 fusion. Figure shows duplicate determinations of one patient specimen.

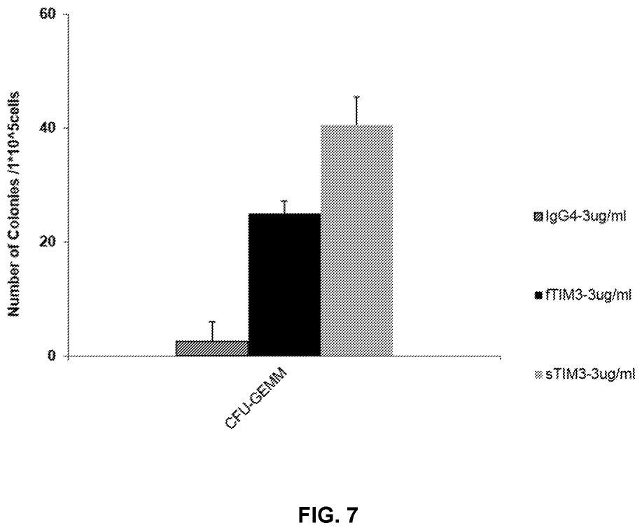

FIG. 8 is a bar graph showing results of a hematopoietic colony forming assay after treatment to measure the restoration of specific progenitors after treatment with IgG4 control or sTIM3 fusion or fTim3 fusion. Figure shows duplicate determinations of one patient specimen.

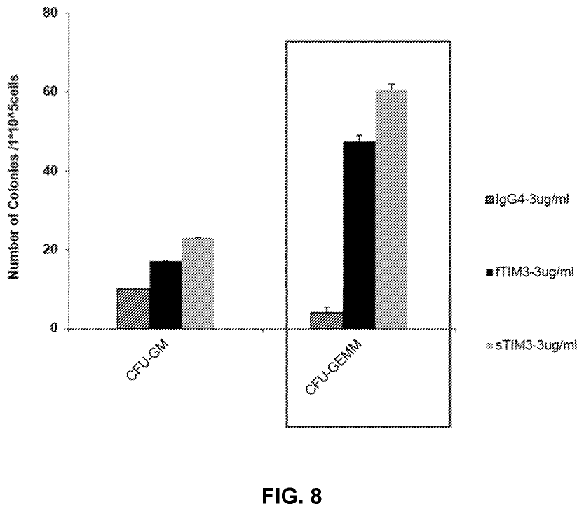

FIG. 9 contains images from one representative experiment showing overall image of colony formation by both low or high dose treatment of MDS-BM with full-length or shorten version of Tim3 chimeric protein.

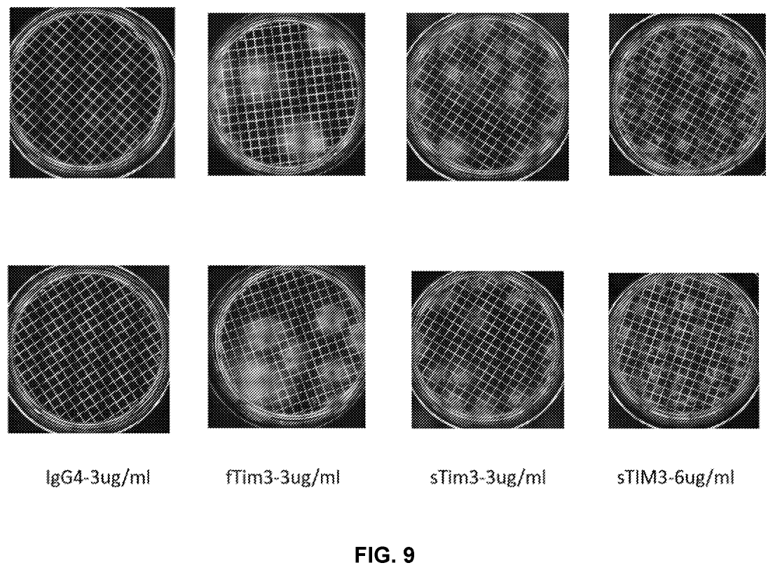

FIG. 10 contains images from another representative experiment showing overall image of colony formation by both low or high dose treatment of MDS-BM with full-length or shorten version of Tim3 chimeric protein.

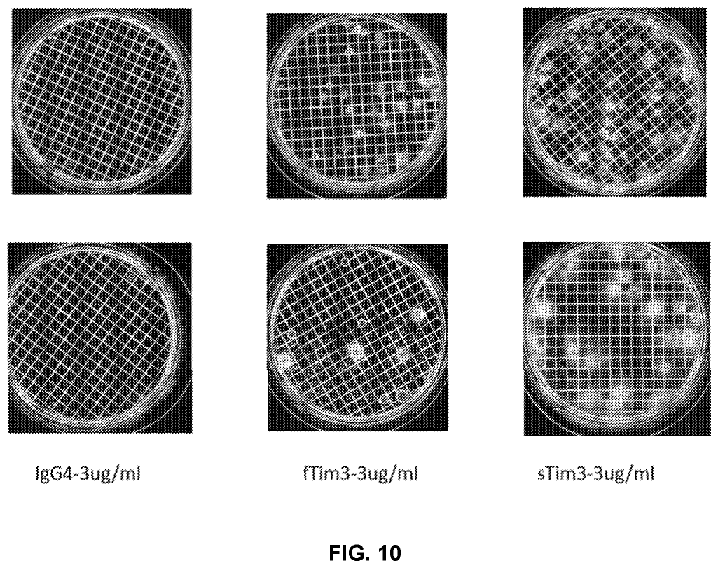

FIG. 12 contains high power images to show typical GEMMs colonies after treatment with sTIM3 chimeric protein.

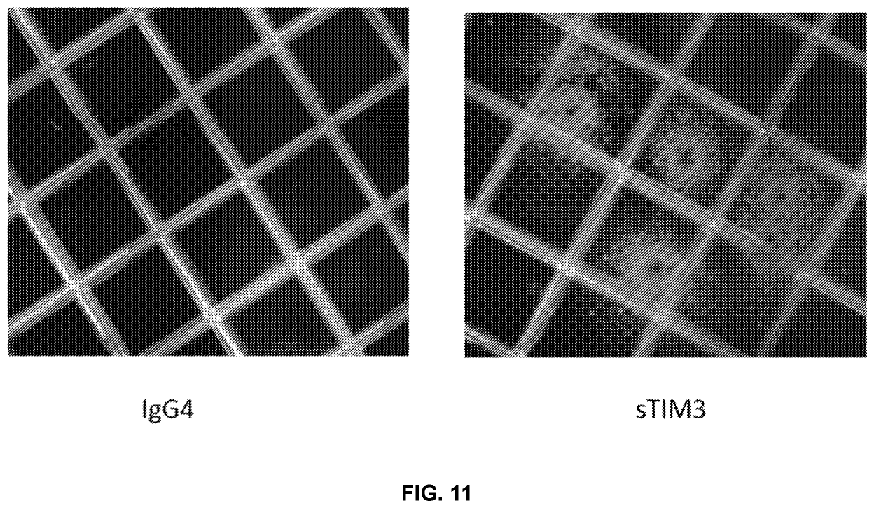

FIG. 12 contains high power images from one representative experiment of GEMM under high power.

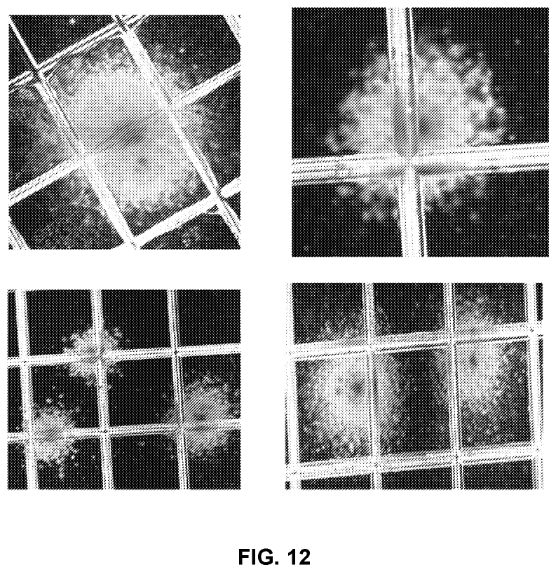

FIG. 13 contains high power images from patients of GEMM under high power.

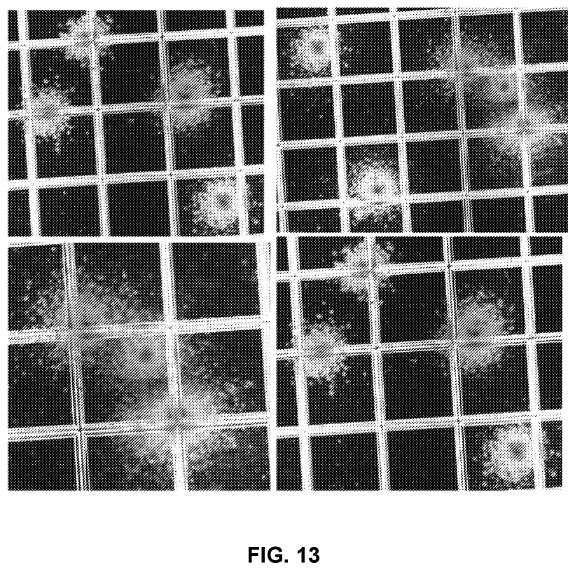

FIG. 14 is a bar graph showing galectin 9 levels measured in MDS plasma specimens.

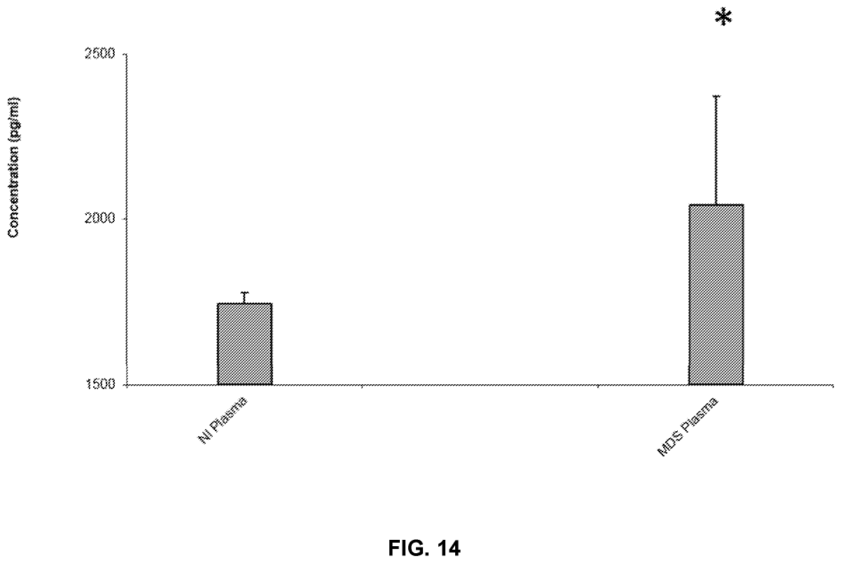

FIG. 15 contains images of immunostaining of healthy bone marrow with both sTim-3-fusion and fTim-3-fusion indicating there is no binding of Tim3-fusion on healthy bone marrow cells.

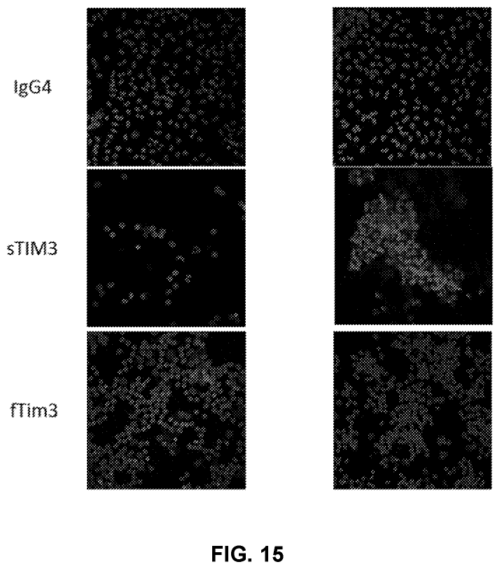

FIG. 16 contains images from one representative experiment showing immunostaining of primary bone marrow mononuclear cells fresh isolated from MDS patients with both sTim-3-fusion and fTim-3-fusion, indicating there is direct binding of Tim3-fusion proteins on bone marrow cells.

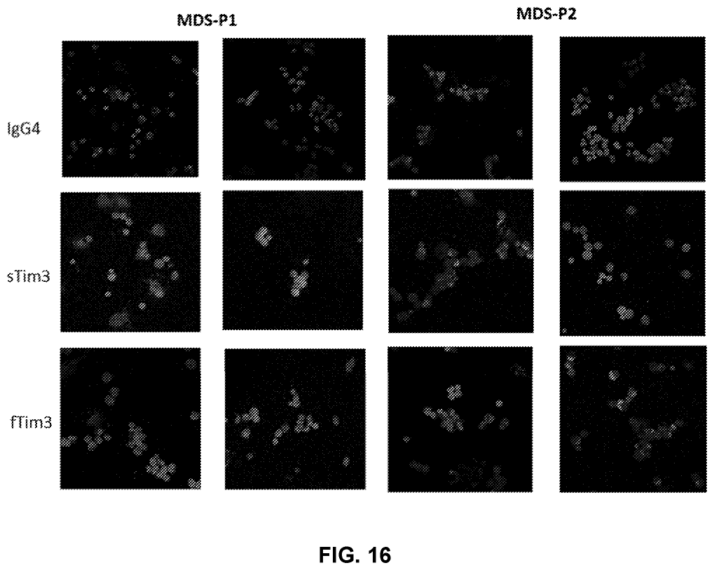

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “protein domain” refers to a portion of a protein, portions of a protein, or an entire protein showing structural integrity; this determination may be based on amino acid composition of a portion of a protein, portions of a protein, or the entire protein.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

Chimeric Tim3 Receptor

The T-cell immunoglobin mucin-3 (TIM-3) receptor, and its main ligand galectin-9 (Gal-9), regulates self-renewal of human leukemic stem cells (LSCs) through co-activation of both NF-κB and β-catenin signaling. While most of the studies on this protein have focused on exhausted T cells, and current therapeutic strategies are developing antibodies to block this receptor in this compartment. However, after induction by an inflammatory stimulus, TIM-3 can also be expressed on mast cells, monocytes, microglia, splenic cDCs, and human circulating cDCs. Notably, TIM-3 and galectin-9 were also found to be expressed on the plasma membrane of LSCs in all FAB types of AML with the exception of APL and in MDS, while absent on normal (non-malignant) HSCs. These findings were also linked to leukemic transformation of a variety of preleukemic disorders, including MDS and MPNs, in which TIM3 surface expression increased in the CD34+CD38− population accompanied by out-competition of normal HSCs with progression to AML to establish clonal dominance. However, the role of TIM3 is strongly related to its ligands, including Galectin-9 (Gal-9). Gal-9 levels were significantly increased in sera from AML patients as well as mice xenografted with primary human AML specimens, and neutralization of Gal-9 has been demonstrated to inhibit AML xenograft reconstitution. Therefore, targeting the ligands of TIM-3 could be an attractive strategy in MDS and AML. However, focusing on Gal-9 only as a target could be very limited due to the multiple ligands of TIM-3 that may play a key role in leukemia.

Therefore, a chimeric-Tim3 receptor was designed that comprised the ectodomain of TIM-3 and the Fc portion of human IgG4, which has reduced Fc receptor binding affinity. Surprisingly, this fusion protein markedly stimulated colony formation of primary MDS bone marrow specimens treated ex vivo. It directly binds to human MDS specimens indicating that the fusion protein interacts with a potential protein ligand directly in LSCs.

NGS mutational analysis was conducted to confirm whether these colonies after treatment with Tim-3-fusion were distinct from original malignant clones. Results indicated these Tim-3-fusion stimulated cells do not contain mutations that were present in the MDS malignant clone. For example, one patient had GATA3 mutations in his/her pathological diagnosis report, however the Tim-3-fusion stimulated colonies did not contain a GATA3 mutation.

Disclosed herein are soluble TIM-3 receptor fusion proteins. More specifically, disclosed is a chimeric fusion protein comprising a TIM-3 receptor moiety covalently linked to at least one fusion moiety.

TIM-3 Receptor Moieties

A TIM-3 receptor moiety comprises at least a portion of a TIM-3 receptor extracellular domain (ectodomain) that can be covalently linked to a fusion moiety. In some embodiments, the TIM3 ectodomain comprises at least the IgV domain. In some embodiments, the TIM3 ectodomain further comprises the mucin domain, the transmembrane domain, the cytoplasmic domain, or any combination thereof. In some embodiments, the TIM3 ectodomain lacks the mucin domain, the transmembrane domain, the cytoplasmic domain, or any combination thereof. The fusion protein can further comprise a signal peptide for secretion of the protein.

The TIM-3 receptor of different species has been cloned and characterized. Preferred polypeptide sequences of the TIM-3 receptor moiety are those corresponding to the wild-type human TIM-3 receptor, which are disclosed in the accession numbers: AAL65158.1, AAL65157.1, AF450242.1, and Q8TDQ0.3. Therefore, in some embodiments, the TIM-3 receptor moiety comprises at least a portion of amino acid sequence: MFSHLPFDCVLLLLLLLLTRSSEVEYRAEVGQNAYLPCFYTPAAPGNLVPVCWGKG ACPVFECGNVVLRTDERDVNYWTSRYWLNGDFRKGDVSLTIENVTLADSGIYCCRI QIPGIMNDEKFNLKLVIKPAKVTPAPTLQRDFTAAFPRMLTTRGHGPAETQTLGSLP DINLTQISTLANELRDSRLANDLRDSGATIRIGIYIGAGICAGLALALIFGALIFKWYSHS KEKIQNLSLISLANLPPSGLANAVAEGIRSEENIYTIEENVYEVEEPNEYYCYVSSRQ QPSQPLGCRFAMP (SEQ ID NO:9), or a variant thereof capable of binding a TIM-3 ligand.

As will readily be understood by one of ordinary skill in the art, sequences homologous to those preferred sequences are also included within the definition. Homologous sequences may contain modifications (such as one or more conservative substitutions, deletions, additions, or alterations produced by mutated cells). “Conservative substitutions” of a residue in a reference sequence are substitutions that are physically or functionally similar to the corresponding reference residue, e.g., that have a similar size, shape, electric charge, chemical properties, including the ability to form covalent or hydrogen bonds, or the like.

Fusion Moieties

A fusion moiety may be any polypeptide entity that can be linked to a TIM-3 receptor moiety described herein to produce a soluble fusion protein as provided herein. A fusion moiety may be selected to confer any of a number of advantageous properties to the fusion proteins. A fusion moiety may be selected to provide increased expression of the recombinant fusion protein. A fusion moiety may, alternatively or additionally, facilitate purification of the fusion protein by, for example, acting as a ligand in affinity purification. A proteolytic cleavage site may be added to the recombinant protein so that the desired polypeptide sequence can ultimately be separated from the fusion moiety after purification. Proteolytic enzymes include, for example, factor Xa, thrombin, enteroprotease, and enterokinase. A fusion moiety may also be selected to confer an improved stability to the fusion protein, when stability is a goal. Other advantageous properties include, but are not limited to, enhanced solubility, increased immunogenicity, detectability (e.g., by chemiluminescence or fluorescence), and easy administration to a patient (e.g. by direct injection).

Any of a variety of polypeptide moieties may be employed as a fusion moiety in accordance with the present invention. Suitable fusion moieties for use in the present fusion protein include, for example, antibodies or portions thereof, and polyhistidine tags (e.g., six histidine residues), that allow for the easy purification of the fusion protein on a nickel chelating column (J. Porath, Prot. Exp. Purif. 1992, 2: 263-281). Glutathione-S-transferase (GST), maltose E binding protein, or protein A are other suitable fusion moieties that can be fused to a TGF-β type III receptor moiety using commercial fusion expression vectors such as pGEX (Amrad Corp., Melbourne, Australia; D. B. Smith and K. S. Johnson, Gene, 1988, 67: 3140), pMAL (Hew England Biolabs, Beverly, Mass.), and pRIT5 (Pharmacia, Piscataway, N.J.), respectively.

In certain embodiments, the fusion-moiety comprises all or a portion of the constant region of an immunoglobulin. In some embodiments, the fusion moiety comprises the Fc tail of human IgG; such as IgG1, IgG2, IgG3, or IgG4. The Fc tail of human IgG2 promotes the expression of the recombinant protein and allows for an easy purification by protein-A column chromatography. A fusion protein of the invention comprising the Fc tail of human IgG4 has a long half-life, can be administered by direct injection, and does not elicit an immune response.

Fusion proteins, also known as chimeric proteins, are proteins created through the joining of two or more genes which originally coded for separate proteins. Translation of this fusion gene results in a single polypeptide with function properties derived from each of the original proteins. Recombinant fusion proteins can be created artificially by recombinant DNA technology for use in biological research or therapeutics. Chimeric mutant proteins occur naturally when a large-scale mutation, typically a chromosomal translocation, creates a novel coding sequence containing parts of the coding sequences from two different genes.

The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability. Thus, any of the herein disclosed functional domains can be used to design a fusion protein.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein. The protein can be engineered to include the full sequence of both original proteins, or only a portion of either.

Often linker (or “spacer”) peptides are also added which make it more likely that the proteins fold independently and behave as expected. Especially in the case where the linkers enable protein purification, linkers in protein or peptide fusions are sometimes engineered with cleavage sites for proteases or chemical agents which enable the liberation of the two separate proteins. This technique is often used for identification and purification of proteins, by fusing a GST protein, FLAG peptide, or a hexa-his peptide (aka: a 6×his-tag) which can be isolated using nickel or cobalt resins (affinity chromatography).

Suitable linkers for fusion proteins antibodies are known in the art. In some embodiments, the linker comprises the amino acid sequence GGSGGSGGS (SEQ ID NO:8).

Nucleic Acids and Vectors

Also disclosed are polynucleotides and polynucleotide vectors encoding the disclosed chimeric polypeptides.

Nucleic acid sequences encoding the disclosed polypeptides, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding polypeptides is typically achieved by operably linking a nucleic acid encoding the polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a chimeric polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

Pharmaceutical Composition

Also disclosed is a pharmaceutical composition comprising a fusion protein disclosed herein in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. P P. Gerbino, Lippincott Williams & Wilkins, Philadelphia, Pa. 2005. Typically, an appropriate amount of a pharmaceutically-acceptable salt is used in the formulation to render the formulation isotonic. Examples of the pharmaceutically-acceptable carrier include, but are not limited to, saline, Ringers solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers dextrose, dextrose and sodium chloride, lactated Ringers, or fixed oils. Intravenous vehicles include fluid and nutrient replenishers, electrolyte replenishers (such as those based on Ringers dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Methods of Treatment

Also disclosed is a method for treating a hematological cancer, such as a myelodysplastic syndrome (MDS) or acute myeloid leukemia (AML), in a subject by administering to the subject a therapeutically effective amount of the disclosed pharmaceutical composition. The method can further involve administering to the subject lenalidomide, or an analogue or derivative thereof.

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The compositions disclosed herein may be administered prophylactically to patients or subjects who are at risk for a hematological cancer. Thus, the method can further comprise identifying a subject at risk for a hematological cancer prior to administration of the herein disclosed compositions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

In some embodiments, the molecule is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of molecule administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

Methods

Clone and Produce Tim-3 Fusion Chimeric Protein
1. Design primers

TABLE 1
Primers

| name | Sequences 5′-3′ |
|---|---|
| Tim3P1-EcoRI- | CAC TTG TCA CGA ATT CG ATG T TTT CAC ATC TTC CCT |
| IgG4-Fc2-F | TTG ACT G (SEQ ID NO: 10) |
| sTim3P2-linker-R | TAA AGC GCC GGA GCC ACC GCT GCC GCC AGA ACC |
| | ACC TGG TTT GAT GAC CAA CTT CAG (SEQ ID NO: 11) |
| sTIM3P3-linker-F | GGT GGT TCT GGC GGC AGC GGT GGC TCC GGC GCT |
| | TTA ATT TTC AAA TGG (SEQ ID NO: 12) |
| TIM3P4-BgI II- | GGC ATG GGG GAG ATC TTG GCA TTG CAA AGC GAC |
| IgG4-Fc2-R | (SEQ ID NO: 13) |

2. PCR using pCDEF3-hTIM3 plasmid from Addgene as template. (AF450242.1)
3. Restrict pFuse-IgG4-Fc2 with restriction enzymes EcoR I and Bgl II.
4. Ligate PCR products and pFuse-IgG4-Fc2 using in-fusion cloning kit from Clonetech.
5. Transform ligation mix into Stellar component cells and culture in LB agar plate overnight.
6. Pick up single colony to amplify and isolate plasmid, and sequence.
7. Transfect Tim3-pFuse-IgG4-Fc2 plasmid into CHO cell with lipofectamine-2000 reagent, select with zeocin 100 ug/ml and get stable cells.
8. Collect supernatant and cell lysate, add protein A/G beads to rotate overnight.
9. Wash beads with PBS, and add elution buffer to elute Chimeric protein.

Colony Formation Assay:
1. Count cells 1*10\hat{ }6/ml, add 200 ul into 3 ml MethoCult medium.
2. Add 6 ug/ml IgG4, sTim3 or fTim3 into 3 ml MethoCult medium.
3. Mix, and separate into 2 wells/3 ml on 6-well plate. Culture 12 days and count colony.

Immunostaining Assay:
1. Count 3×10⁴/slide onto slide, cytospin 300 rpm for 3 minutes.
2. Fix slide Cytofix Fixation Buffer and incubate at 37 C for 10 min.
3. Permeabilize the cells for 15 min at RT using a 0.1% Triton-X100 in 2% BSA/PBS solution. Wash with PBS
4. Block for 1 h at RT using a 2% BSA/PBS solution. Wash with PBS.
5. Incubate primary Antibody 1 hour. Wash with PBS
6. Incubate secondary antibody for 1 hours. Wash with PBS.
7. Add 1 drop of DAPI and cover with a coverslip.
8. Keep at 4 C until ready to image.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A chimeric polypeptide, comprising a TIM3 ectodomain and an Fc portion of an immunoglobulin.

2. The polypeptide of claim 1, wherein the TIM3 ectodomain comprises at least the IgV domain.

3. The polypeptide of claim 2, wherein the TIM3 ectodomain further comprises the mucin domain, the transmembrane domain, the cytoplasmic domain, or any combination thereof.

4. The polypeptide of claim 1, wherein the TIM3 ectodomain lacks the mucin domain, the transmembrane domain, the cytoplasmic domain, or any combination thereof.

5. The polypeptide of claim 1, further comprising signal peptide.

6. The polypeptide of claim 1, wherein the polypeptide is defined by the formula:
SP-IgV-Cyto-Fc
wherein “SP” represents a signal peptide,
wherein “IgV” represents an IgV domain of TIM3,
wherein “Mucin” represents a mucin domain of TIM3,
wherein “TM” represents an transmembrane domain of TIM3,
wherein “Cyto” represents a cytoplasmic domain of TIM3,
wherein “Fc” represents an Fc portion of an immunoglobulin, and
wherein “-” represents a linker peptide or peptide bond.

7. The polypeptide of claim 1, wherein the polypeptide is defined by the formula:
SP-IgV-Mucin-TM-Cyto-Fc
wherein “SP” represents a signal peptide,
wherein “IgV” represents an IgV domain of TIM3,
wherein “Mucin” represents a mucin domain of TIM3,
wherein “TM” represents an transmembrane domain of TIM3,
wherein “Cyto” represents a cytoplasmic domain of TIM3,
wherein “Fc” represents an Fc portion of an immunoglobulin, and
wherein “-” represents a linker peptide or peptide bond.

8. A pharmaceutical composition comprising the molecule of claim 1 in a pharmaceutically acceptable carrier.

9. A method for treating a hematological cancer in a subject, comprising administering to the subject a therapeutically effective amount of the pharmaceutical composition of claim 8.

10. The method of claim 9, wherein the hematological cancer comprises a myelodysplastic syndrome (MDS).

11. The method of claim 9, wherein the hematological cancer comprises an Acute Myeloid Leukemia (AML).

12. A method for enhance hematopoiesis in a subject, comprising administering to the subject a therapeutically effective amount of the pharmaceutical composition of claim 8.


SEQUENCE LISTING (13 sequences)

SEQ ID NO: 1 - protein, 301 aa, Artificial Sequence
  Synthetic Construct
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 2 - protein, 222 aa, Artificial Sequence
  Synthetic Construct
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPGGSGGSGGSGALIFKWYSHSKEKIQNLSLISLANLPPSGLANAVAEGIRSEENIYTIEENVYEVEEPNEYYCYVSSRQQPSQPLGCRFAMP

SEQ ID NO: 3 - protein, 21 aa, Artificial Sequence
  Synthetic Construct
MFSHLPFDCVLLLLLLLLTRS

SEQ ID NO: 4 - protein, 105 aa, Artificial Sequence
  Synthetic Construct
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLK

SEQ ID NO: 5 - protein, 74 aa, Artificial Sequence
  Synthetic Construct
LVIKPAKVTPAPTRQRDFTAAFPRMLTTRGHGPAETQTLGSLPDINLTQISTLANELRDSRLANDLRDSGATIR

SEQ ID NO: 6 - protein, 21 aa, Artificial Sequence
  Synthetic Construct
IGIYIGAGICAGLALALIFGA

SEQ ID NO: 7 - protein, 80 aa, Artificial Sequence
  Synthetic Construct
LIFKWYSHSKEKIQNLSLISLANLPPSGLANAVAEGIRSEENIYTIEENVYEVEEPNEYYCYVSSRQQPSQPLGCRFAMP

SEQ ID NO: 8 - protein, 9 aa, Artificial Sequence
  Synthetic Construct
GGSGGSGGS

SEQ ID NO: 9 - protein, 301 aa, Artificial Sequence
  Synthetic Construct
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 10 - DNA, 43 bp, Artificial Sequence
  Synthetic Construct
cacttgtcacgaattcgatgttttcacatcttccctttgactg

SEQ ID NO: 11 - DNA, 57 bp, Artificial Sequence
  Synthetic Construct
taaagcgccggagccaccgctgccgccagaaccacctggtttgatgaccaacttcag

SEQ ID NO: 12 - DNA, 48 bp, Artificial Sequence
  Synthetic Construct
ggtggttctggcggcagcggtggctccggcgctttaattttcaaatgg

SEQ ID NO: 13 - DNA, 33 bp, Artificial Sequence
  Synthetic Construct
ggcatgggggagatcttggcattgcaaagcgac